(19) United States
(12) Patent Application Publication
(10) Pub. No.: US 2012/0053598 A1
(43) Pub. Date: Mar. 1, 2012

(54) Helical Fasteners and Methods for Deploying Same

(75) Inventor: Albert K. Chin, Palo Alto, CA (US)

(73) Assignee: Pavilion Medical Innovations

(21) Appl. No.: 13/219,542

(22) Filed: Aug. 26, 2011

Related U.S. Application Data
(60) Provisional application No. 61/377,203, filed on Aug. 26, 2010.

(51) Int. Cl. A61B 17/00; A61B 17/03

(52) U.S. Cl. 606/139; 606/151

(57) ABSTRACT

A system is provided for fastening tissue. The system includes a fastening device designed to bias between an actuated state for delivering into tissue and a quiescent state for fastening the tissue. In an embodiment, the fastening device may include a plurality of coils between which tissue can be pinched or fastened. The system also includes a pathway along which the fastening device can be accommodated for subsequent delivery into the tissue, and an activation mechanism along the pathway to bias the fastening device into an activated state for delivery into the tissue. Fastening devices, and methods of fastening are also provided.

### RELATED APPLICATIONS

This application claims priority to and the benefit of U.S. Provisional Application No. 61/377,203, filed Aug. 26, 2010, which is incorporated here by reference.

### TECHNICAL FIELD

This invention relates generally to surgical fasteners, and more particularly, to fastening devices for surgically fastening tissue.

### BACKGROUND

Fasteners have been used in surgical procedures to eliminate the need for suturing, which can be both time consuming and inconvenient. In many applications, the surgeon can use an apparatus to delivery the fasteners. Using fasteners may reduce the time required for the procedure and may further reduce complications associated with the procedure, such as blood loss and trauma to the patient.

Conventional fasteners can be found in various forms, including, for instance, metal staples. These staples are bent by the delivery apparatus to connect tissue together. The staples may use an applicator for delivery. The applicator may be designed to project the conventional staple into tissue as well as to deform the staple so that it is retained against the tissue. In certain applications, access to the body tissue from two opposite directions is required to deform the legs of the staple after they have passed through the body tissue.

Another type of fastener may include two-part fasteners. Two-part fasteners may use a barbed staple in conjunction with a retaining piece to hold the staple in place. For example, the two-part fastener may include a crown or backspan and two barbed prongs which may be engaged and locked into a separate retainer piece. In use, the two-part fastener may be pressed into the body tissue so that the barbs penetrate the tissue and emerge from the other side where they are then locked into the retainer piece. Retainers prevent the staple from migrating from the tissue. The two-part fasteners are generally not removable. Similar to the metal staple, however, the two-part fasteners may require the staple delivery apparatus to have access to both sides of the tissue.

Since conventional fasteners require access to both sides of the tissue, it may not be appropriate or possible to utilize conventional fasteners during certain surgical procedures.

Accordingly, it would be desirable to have an applicator that dispenses a surgical fastener having high surface area for retentive contact with tissue and that can be delivered into body tissue from one direction, while minimizing or reducing the likelihood of migration.

### SUMMARY OF THE INVENTION

A system is provided for fastening layers of tissue or other material during a surgical procedure. The system includes a fastening device designed to bias between an actuated state for delivering into tissue and a quiescent state for fastening the tissue. In an embodiment, the fastening device may include a plurality of coils between which tissue can be pinched or fastened. In the activated state, the coils may be spread apart from one another for delivery into the system, and in the quiescent state, the coils may be compressed toward one another in order to pinch or fasten tissue between adjacent coils. The fastening device may also, in some embodiments, include a coupling mechanism to hold multiple segments of the fastening device in axial alignment and/or a segmentation point to allow the fastening device to be separated into a plurality of segments.

The system may also include a pathway along which the fastening device can be accommodated for subsequent delivery into the tissue. The pathway may be used to direct the fastening device to a site of fastening. An activation mechanism along the pathway may allow the fastening device to be biased into an activated state during delivery into the tissue. Fastening devices, and methods of fastening are also provided.

### BRIEF DESCRIPTION OF DRAWINGS

FIG. 1 shows a fastening device in accordance with an embodiment of the present invention.

FIG. 2 shows a fastening device, in accordance with an embodiment of the present invention, connecting two layers of tissue to one another.

FIG. 3 shows a system for delivering a fastening device in accordance with an embodiment of the present invention.

FIG. 4 shows a fastening device being delivered across two tissue layers in accordance with an embodiment of the present invention.

FIG. 5 shows a delivery device in accordance with an embodiment of the present invention.

FIGS. 6a-6c show a delivery device in accordance with another embodiment of the present invention.

### DESCRIPTION OF SPECIFIC EMBODIMENTS

In accordance with one embodiment of the present invention, fastening devices are provided for retentive contact with tissue and designed to be delivered into the tissue from one direction without needing access from an opposite direction to secure the fastening device to the tissue. In some embodiments, the fastening devices may be used to fasten multiple layers of tissue together and/or may fasten a foreign object (such as a mesh) to tissue. The fastening devices of the present invention may find use in, for instance, securing prosthetic mesh onto the fascial or skin wall of a patient during abdominal hernia repair. It should be appreciated that the device of the present invention can be adapted for securing other items against tissue. As used herein, the term “tissue” generally refers to the fascia, skin or layers of cells that cover muscles and various body organs. However, “tissue” may also refer to any organic or inorganic material that may be fastened.

Fastening Device

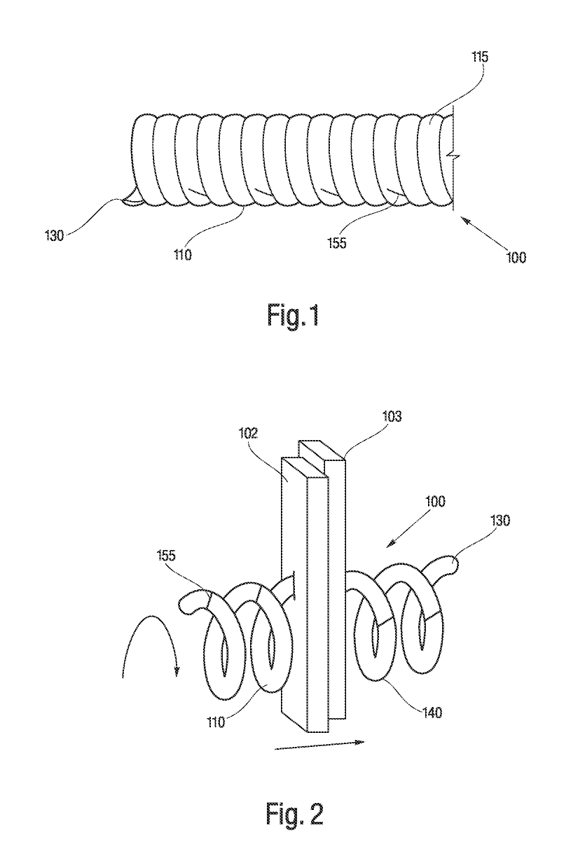

FIGS. 1 and 2 illustrates the fastening device 100 in accordance with one embodiment of the present invention. The fastening device 100 of the present invention includes, in an embodiment, a body portion 110 capable of biasing between an actuated state to facilitate penetration of the device into tissue, and a resting or quiescent state to fasten or connect at least two layers of tissue to one another. The body portion 110 may be designed to penetrate through tissue and secure itself within the tissue.

To facilitate the ability of device 100 to penetrate into or through tissue, the body portion 110 may be provided with sufficient flexibility to permit bending of the device 100, while being sufficiently strong to maintain the device 100 in place. The body portion 110, in an embodiment, may have a length, diameter or thickness of any size, depending on the particular application, as the length, diameter or thickness of the body portion 110 may affect the strength and/or flexibility of the fastening device 100. It should be noted, in an embodiment, the diameter of the body portion 110 may be substantially uniform along its length for ease of delivery. In an alternative embodiment, the diameter of the body portion 110 may vary along its length, depending on the application.

To allow the body portion 110 to be sufficiently flexible the body portion 110 may, in an embodiment, be provided with a framework arranged in a geometric or non-geometric pattern, such as a helix, lattice, or any other suitable pattern. The pattern of the framework, in an embodiment, may provide the necessary strength and/or flexibility to the fastening device 100. For instance, a coil pattern may provide the body with sufficient flexibility.

As shown in FIGS. 1-2, the body portion 110 may be formed from one or more pieces of wire 115. As used herein, “wire” may refer to any type of wire, strand, strut or structure, regardless of cross-sectional dimension (e.g., the cross-section could be circular, oval, or rectangular) or shape, and regardless of material, that may be used to construct a device as described herein. The wire 115 forming the body portion 110, in an embodiment, may have a length, diameter or thickness of any size, depending on the particular application, as the length, diameter or thickness of the wire 115 may affect the strength and/or flexibility of the fastening device 100.

In order to advance and/or penetrate into the tissue, and secure itself to the tissue, the body portion 110, in one embodiment, may be defined by a plurality of coils 140. Coils 140 may be in a helical pattern, so that body portion 110 can bias between an actuated state, where the coils 140 are spread apart from one another, for delivery of the body portion 110 into the tissue. In particular, with coils 140 spread apart from one another, the body portion 110 may be advanced into the tissue in a rotational or screw-like manner. For example, in response to a rotational force or motion, coils 140 may be advanced into or through tissue 102 and/or 103, as shown in FIG. 2. In this position, the coils 140 may be provided with any desirable pitch or gap between adjacent coils 140, so long as the pitch is sufficient to accommodate the thickness of the tissue between adjacent coils 140.

The coils 140 can also be placed in the resting position, i.e. a relaxed or quiescent state, where the coils 140 are compressed toward one another to sandwich the tissue therebetween for retentive or fastening purposes. In the resting or quiescent position, each coil 140 may be substantially compressed against an adjacent coil 140 so as to permit the body portion 110 to approximate its original position. In this way, the coils 140 of body portion 110 can act to secure themselves to the tissue at the site of fastening and/or to hold tissue and other objects between the coils. In particular, adjacent coils 140 can compress towards one another in order to secure tissue therebetween, to hold layers of tissue and other objects together, and to minimize subsequent movement of the device 100 from the site of fastening. In this compressed position, the coils 140 may be adjacent to one another, with substantially no space between adjacent coils 140. Although no space is illustrated, it should be appreciated that a space may exist between adjacent coils 140 due to the presence of tissue or other objects or material between adjacent coils. It should be noted that the coils 140 may be designed with any desired diameter as long as the diameter can permit the device 100 to fit within any suitable trocar, catheter, needle or other delivery mechanism for insertion into the body.

As used herein, the term “coil” may refer to one complete revolution of the wire 115. However, the term “coil” may also refer to a partial revolution, or multiple revolutions of the wire 115. In some embodiments, body portion 110 may have a cylindrical shape and the coils may form a helix. In other embodiments, body portion 110 may have any suitable geometry (e.g. triangle, hexagon, square, etc). In these embodiments, each coil may have the same or a similar shape as the body. It should be noted that the number of coils 140 may vary depending on the length of the body portion 110 and the diameter of body portion 110. The body, and therefore the coils, may have any appropriate diameter depending on the fastening application. The wire or material from which the coils are constructed may also have any sufficient diameter for penetrating into the tissue and fastening tissue or other objects. The diameter may be sufficiently small for penetration into the body and sufficiently large to provide rigidity and strength for fastening. It should be appreciated that while described herein as forming a coil 140, other designs may also be possible as the present invention is not intended to be limited in this manner.

Although described herein as a helical coil, the body portion 110 may be provided with other designs, for instance, a lattice design capable of extending into an activated open state and contracting into a relaxed closed state.

The fastening device 100 of the present invention may also include penetration portion 130 positioned at a distal end of the body portion 100. In one embodiment, the penetration portion 130 allows for the initial penetration of the body portion 100 into the tissue at the site of fastening. In some embodiments, penetration portion 130 may be sharpened or pointed to facilitate penetration of the body portion 110 into the tissue. In the presence of a rotational force applied to body portion 110, penetration portion 130 may act to penetrate the tissue and be advanced into the tissue. the continual advancement of body portion 110 into the tissue can result in movement of the body portion 110 in a direction substantially transverse to a surface of the tissue, as shown in FIG. 2.

As shown in FIGS. 1 and 2, the penetration portion 130 may be an end point of the distal most coil 140. In an embodiment, substantially no spacing may exist between the penetration portion 130 and the first complete coil 140. However, it should be appreciated that a space may exist between the penetration portion 130 and the first complete coil 140, so that the penetration portion 130 does not lie flat against the body portion 110. The penetration portion 130 may be designed, in one embodiment, to be sufficiently sharp to penetrate the skin and direct the body portion 110 into tissue.

In certain instances, it may be desirable to have a body portion 110 provided with an extended length. To increase the length of fastening device 100 of the present invention, fastening device 100 may be provided with a coupling mechanism, such as a bridge, at segmentation point 155, such as a bridge. As shown in FIG. 2, the segmentation point 155 may be used to connect a plurality of bodies 110 of device 100 to form an elongated and substantially continuous structure or body portion 110. In such an approach, the elongated and substantially continuous structure can be provided with any desired length, depending on the number of bodies 110 coupled to one another.

Alternatively, coupling mechanism may be used to connect individual coils to one another to provide body portion 110. In such and approach, body portion 110 can be provided with any desired length, depending on the number of coils 140 coupled to one another. With such a design, the specific length of body portion 110 that may be fastened into tissue can be more precisely controlled.

It should be appreciated that while described as a bridge, any coupling mechanism, for example adhesives or other attachment mechanisms known in the art, can also be used.

In addition, the coupling mechanism, in one embodiment, may be provided with any size, geometric shape, length, or diameter desired so long as the dimensions of the coupling mechanism minimize interference with the delivery of the fastening device 100. In addition, coupling mechanism can be formed from any material that is sufficiently strong to hold adjacent coils 140 together and is also capable of being severed.

In an embodiment of the present invention, segmentation points 155 may be regularly situated along body portion 110 in order to divide body portion 110 into a plurality segments. In this way, segmentation points 110 may allow body portion 110 to be broken or segmented into various or desired lengths. Segmentation points 155, in one embodiment, may be situated on every coil, or may skip one or more coils, to provide segments of different sizes and allow control over the length of the segments. Segments of various sizes may be desired for different applications, different surgeries, different tissue types, etc. Segmentation points 155 may be regularly situated along body portion 110, or may be provided in any other regular or irregular pattern along body portion 110, depending upon the application.

Since the device 100 may be implanted within tissue of a human or animal, the device 100, and/or portions of the device 100, may be made from a material that is biocompatible. The biocompatibility of the material may help minimize occurrence of adverse reactions due to implantation of the device 100 within the body. Also, device 100 and coils 140 may be made from a bioresorbable material so that it may be left in the body at its site of fastening, and subsequently absorbed by the body. For example, in some embodiments, the device 100 can be made entirely or partially from material that is bioresorbable, or biodegradable, or a combination thereof. In such instances, the device 100 may be entirely or partially absorbed by the vessel or may be degraded after a certain period of time has elapsed, and would eliminate the need for manual removal of the device 100.

In an embodiment, the material from which the body portion 110 may be formed includes metal, metal alloy, polymer, molded plastic, metal-polymer blend, or a combination thereof. The type of material may affect the strength and/or flexibility of the device 100. Examples of suitable materials include shape memory material, stainless spring steel, superelastic metal such as Nitinol, rigid plastic such a polycarbonate, Ultem, or LCP (liquid crystal plastic), or rigid absorbable compounds such as PGA (polyglycolic acid). Other suitable materials include gold, platinum, tungsten, nickel-titanium alloy, Beta III Titanium, cobalt-chrome alloy, cobalt-chromium-nickel-molybdenum-iron alloy, Elgiloy, L605, MP35N, Ta-10W, 17-4PH, Aeromet 100, polyethylene terapthalate (PET), polytetraflouroethylene (PTFE), polyurethane (nylon) fluorinated ethylene propylene (FEP), polyurethane, polypropylene (PP), polyvinylchloride (PVC), polyether-ester, polyester, polyamide, elastomeric polyamides, block polyamide/ethers, polyether block amide (PEBA), silicones, polyethylene, polyether-ether ketone (PEEK), polyimide (PI), polyetherimide (PEI), tantalum, tungsten, or any other suitable material that is biocompatible and that is capable of being expanded in the manner described above. The device 100 may also include an anti-thrombogenic coating such as heparin (or its derivatives), urokinase, or PPack (dextrophenylalanine proline arginine chloromethylketone) to prevent thrombosis or any other adverse reaction from occurring at the site of insertion.

Delivery Mechanism

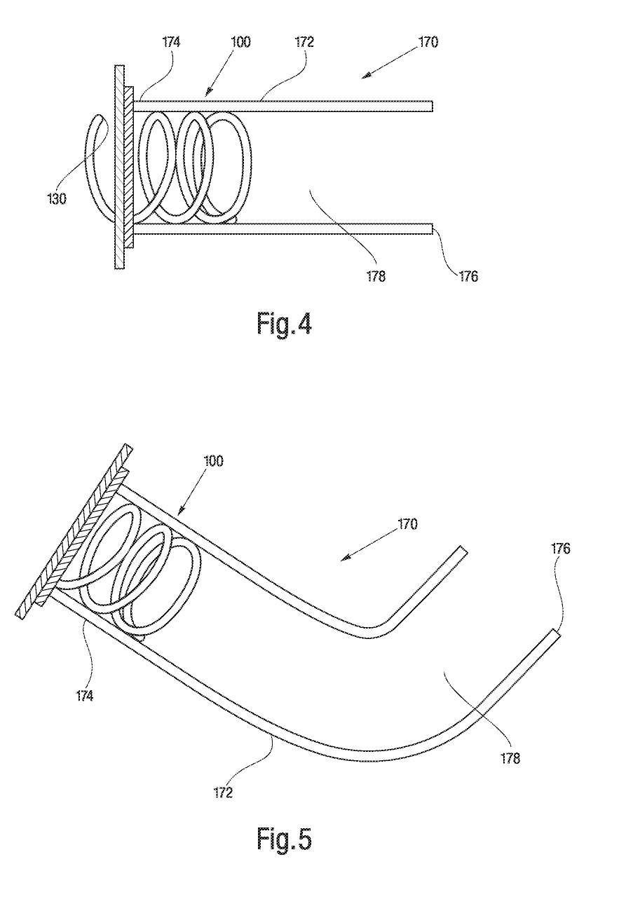

Now referring to FIG. 4, a delivery mechanism 170 may be used for delivering the device 100 to a site of fastening. The delivery mechanism 170, in one embodiment, can include a tube 172 within which device 100 may be accommodated. Tube 172 may have a delivery end 174, an opposing end 176, and a passageway 178 between the ends. With such a design, device 100 may be advanced from opposing end 176 through the passageway 178 and exit the delivery end 174.

In one embodiment, the tube 172 can be made from any material capable of directing it to a site of delivery. To that end, tube 172 may be formed from a substantially flexible or malleable material so as to allow bending or deformation of the tube 172 during delivery, as shown in FIG. 4. Examples of materials that are substantially flexible or malleable include metals, plastics, ceramics, or any other materials that can allow deformation of its shape.

In another embodiment, the tube 172 may be formed from a shape memory material to allow the tube 172 to revert to its substantially straight shape. For instance, during certain stages of delivery, there may be an area where the tube 172 may be deformed or bent. Once cleared of that area, the shape memory of the device 100 may revert the device 100 to its substantially straight shape, thereby allowing the tube 172 to revert to its substantially straight shape for ease of movement subsequently.

It should be appreciated that the tube 172 may be provided with any shape desirable, depending on the particular application, as the shape of the tube 172 may affect the ability of the tube 172 to deliver the device 100 to a site of delivery. For instance, tube 172 may be tubular in shape. Of course, other shapes can be used as the present invention is not intended to be limited in this manner. It should be appreciated that tube 172 can have any shape desired as long as the tube 172 can fit within and be advanced through body.

The tube 172, in another embodiment, may have a length sufficient to accommodate the length of the fastening device 100. It should be appreciated that the length of the tube 172 should permit the tube 172 to be inserted into a body and advanced through the body to a site of delivery.

The tube 172, in another embodiment, may have a diameter sufficient to accommodate delivery of fastening device 100. In some instances, the diameter of the delivery mechanism 170 may remain substantially uniform throughout. If desired, however, the diameter of the delivery mechanism 170 may vary, as necessary.

In certain situations, it may be desirable to minimize bending of the tube 172 during delivery. In such a case, tube 172 may be formed from a substantially hard material. In other cases, the tube 172 may be partially formed from a flexible or malleable material and partially formed from a hard material. For example, FIG. 5 shows that the delivery end 174 may be formed from a hard material while the remaining portion of the tube 172 may be formed from a flexible or malleable material.

Since the tube 172 is designed to be inserted into human or animal tissue, the tube 172, in an embodiment, can be made from a material that is biocompatible. The biocompatibility of the material may help minimize occurrence of adverse reactions due to use of the tube 172 within the body. The tube 172 may further include a coating on an outer surface to reduce friction between the tube 172 and the body upon insertion. Likewise, the tube 172 may include a coating on an inner surface to reduce friction during deployment of the system 100 situated within the tube 172.

In one embodiment, the delivery end 174 can be designed to permit device 100 to be inserted into delivery mechanism 170. To that end, the delivery end 174 may be sufficiently sized to permit the body portion 110 and the coils 140 to be securely positioned within. It should be appreciated that while described as a tube, the tube 172, of course, may have any other geometric shape.

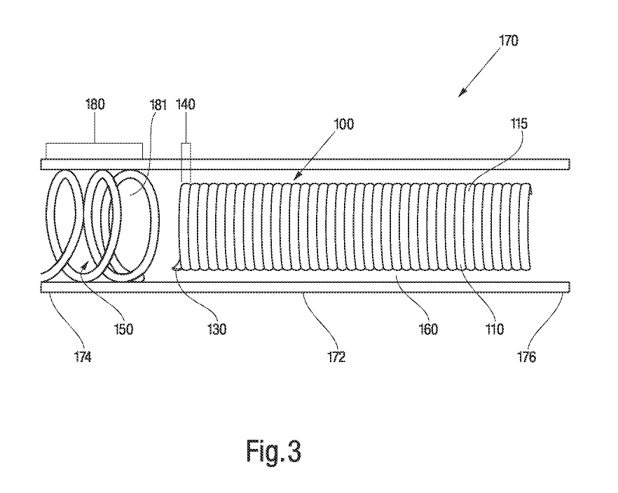

In order to bias the device 100 into an activated position, the delivery mechanism 170 may be provided with an activation mechanism for spreading coils 140 apart, as device 100 is directed into tissue, in order to facilitate penetration of device 100 across or into the tissue. In an embodiment, the activation mechanism for spreading the coils 140 may include spring tracks 180 at its delivery end 174, as shown in FIG. 3, for spreading coils 140 apart from each other while guiding delivery mechanism 170 into tissue.

The spring tracks 180, in an embodiment, may be designed to receive the penetration portion 130 of device 100 and may act as a guide to direct the device 100 into tissue. In an embodiment, the spring tracks 180 may be hollow and may be provided with a passageway 181 through its interior so that body portion 110 may be rotated along the passageway 181 through spring tracks 180 for delivery. Accordingly, the spring tracks 180 may be provided with an inner diameter sufficient to accommodate the body portion 110 of device 100. The spring tracks 180 may also be provided with an open pitch, as shown in FIG. 2, so as to bias the coils 140 from their resting position 160 to an actuated position 150, where the coils 140 are spread apart from one another, as the body portion 110 rotates through the tracks and is delivered into, through, or across tissue. The spring tracks 180, in one embodiment, may be helical to accommodate the device 100. It should be appreciated that while described as helical, the spring tracks 180 may have other designs as well so long as the design can act to bias the body portion 110 of the device 100.

Alternatively, the delivery mechanism 170 may be provided with grooves or tracks (not shown) along its interior surface at delivery end 174 of the tube 172 in order to guide device 100 into the tissue to be fastened. As the coils 140 travel through the grooves situated at the delivery end 174, the grooves may be designed to bias the coils 140 from their resting position 160 to an actuated position 150 where the coils are spread apart. To bias the coils 140, the grooves may be sufficiently spaced to pull the coils 140 apart and allow the coils 140 to be directed into the tissue. The grooves may further define the pitch of the coils 140. In an embodiment, the grooves may have any spacing desired.

As a further alternative, delivery mechanism may be provided with a helical tube (not shown) situated at the delivery end 172. The helical tube may be a hollow tube into which the fastening device may be directed for biasing into an actuated position.

The delivery mechanism 170, in accordance with an embodiment, may also be provided with a deploying mechanism (not shown) from which sufficient force can be applied to advance and/or rotate device 100 along tube 172 and/or spring tracks 180. In one embodiment, the deploying mechanism can be situated at end 176 of tube 172 opposite the delivery end 174. Of course, other locations for the deploying mechanism are possible as long as sufficient force can be applied to deploy the device 100 from the tube 172. In one embodiment, the deploying mechanism can be any mechanical design sufficient to advance the device 100. The deploying mechanism may additionally or alternatively be designed to allow air pressure to enter passageway 178. In another embodiment, the deploying mechanism may further or alternatively be designed to allow fluid or liquid pressure to enter the passageway 178. Of course, other designs of the deploying mechanism may also be possible as the present invention is not intended to be limited in this manner.

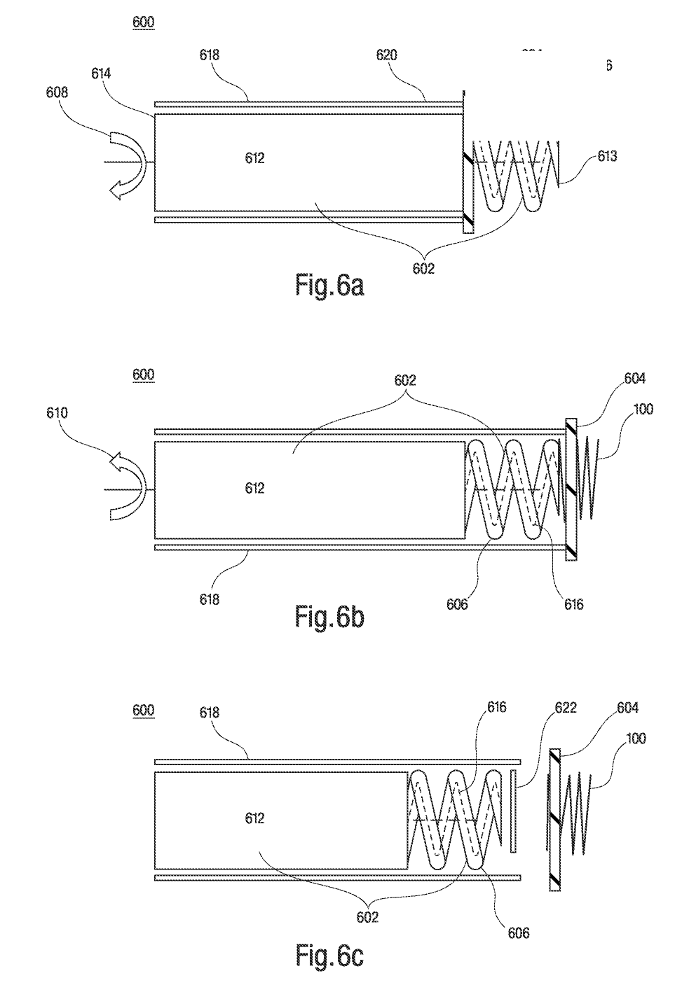

In another embodiment, a delivery device 600 as shown in FIGS. 6a-6b may be provided. Delivery device 600 may include a driver mechanism 602 for penetrating into tissue 604 and delivering fastening device 100 to the site of fastening. Driver mechanism 602 may include, at its distal end, a helical member 606 for penetration into tissue 604 in response to a rotational force. As shown in FIG. 6a, in the presence of a rotational force (as shown by arrow 608), driver mechanism 602 may rotate helical member 606 into tissue 604. Similarly, helical member 606 may be withdrawn or rotated out of the tissue upon exertion of a rotational force in an opposite direction (as shown by arrow 610), as shown in FIG. 6b.

To facilitate penetration of the helical member 606 into tissue 604, helical member 606, in an embodiment, may be provided with penetration portion 612. Penetration portion 612 may, in an embodiment, be sufficiently pointed or sharp so that it can facilitate penetration into or through tissue 604.

Helical member 606, in accordance with an embodiment, may have any desired shape depending upon the surgical application. For example, helical member may have a diameter sufficiently small for penetration into tissue 604, yet sufficiently large for delivery of fastening device 100. The coils of helical member may also have any desired diameter depending upon the surgery or area of implantation. The coils may also have any pitch, grade, or length desired for penetration into tissue 604. In some embodiments, helical member 606 may be adjustable in pitch, length, depth, or any other characteristic to facilitate penetration to a desired depth. In one embodiment, the helical member 606 may be provided with the ability to have its length adjusted by, for instance, allowing it to rotate into or further from the distal end of the driver mechanism 600.

In some embodiments, helical member 606 may be sufficiently rigid for penetration into hard tissue, such as bone or cartilage. Of course, should flexibility be desired, helical member 606 may be provided with such a characteristic. To provide helical member 606 with rigidity, helical member 606 may be constructed from a hard or strong material, such as a metal or molded plastic. Since helical member 606 is designed to be used within a body of a human or animal, helical member 606 may be also constructed from a biocompatible material appropriate for surgery.

Driver mechanism 602 may also include handle 612, which may be coupled to helical member 606 so that a rotational force applied to handle 612 may be transferred to helical member 606. In one embodiment, handle 612 may be provided with flexibility so that it can aid in directing helical member 606 and fastening device 100 to a site of fastening. To the extent desired, handle 612 may have any desired length or diameter for ease of handling.

In one embodiment, driver mechanism 602 may further include pathway 616 extending from the handle 612 through the helical member 606 in order to accommodate fastening device 100 therealong. In that way, fastending device 100 can be directed from the handle 612 into the helical member 606 for subsequent delivery into tissue 604. To access pathway 616, handle 612 may be provided with opening 614 at the proximal end of handle 612. Pathway 616, in an embodiment, may approximate the slope of the helical member 606 so that as fastening device 100 is directed therealong, device 100 can be biased into an actuated state. As shown in FIGS. 6a-c, pathway 616 (and helical member 606) may have a pitch or grade configured to spread the coils of fastening device 100 apart as fastening device 100 is advanced into pathway 616. Pathway 616 (and helical member 606) may have a sufficiently small diameter for penetration into tissue 604, and sufficiently large diameter for delivering fastening device 100 to the site of fastening.

It should be appreciated that with fastening device 100 accommodated in pathway 616, helical member 606 may be rotated or driven into or across tissue 604 in order to advance fastening device 100 therewith.

Once helical member 606 has penetrated across the tissue or to a desired depth, driver 602 may be counter-rotated from the tissue, while leaving the device 100 in place. To allow device 100 to remain in place and not be removed along with the helical member 606 or driver 602, driver 602 may be provided with a locking mechanism (not shown). The locking mechanism may be situated anywhere along driver 602 and/or delivery device 600. Fastening device 100 may also be held in place manually, from the proximal end of handle 602, to prevent rotation of the device 100 along with the helical member 606. In other words, when locked in place, fastening device 100 may remain within the tissue as driver mechanism 602 is rotated out and withdrawn from the tissue. As helical member 606 is withdrawn, fastening device 100 may revert to its resting or quiescent state in order to fasten about tissue 604. As shown in FIG. 6b, the coils of portion 621 of fastening device 100 once released from the helical member 606, revert to their compressed state in order to pinch or hold tissue 604 between adjacent coils.

To aid in directing driver mechanism 602 to the site of fastening, delivery device 600 may also include a guide 618. Guide 618, in one embodiment, may be a tube, such as a catheter or trocar, along which driver 602 can be advanced. Tube 618 may have an opening at its distal end 620 through which helical member 606 can engage the tissue. Like handle 612, tube 618 may be flexible and/or made of a shape memory so that it can more easily direct driver mechanism 602 to the surgical site. Tube 618 and handle 612 may have any desired length and diameter for accommodating driver mechanism 602. Tube 618 may also be constructed from a biocompatible material for use during surgery.

To minimize leaving unnecessary length of device 100 at the site of fastening after removal of driver mechanism 602, delivery device 600 may be provided with a cutting apparatus 622, as shown in FIG. 6c. The cutting apparatus 622 can be used to cut any excess length from the segment of fastening device 100 that has been delivered into tissue 604. Cutting apparatus 622 may be an edge, a blade, a scissor, or any other cutting device capable of separating a portion of fastening device 100 that has been delivered into tissue 604. In an embodiment, cutting apparatus 622 may be coupled to tube 618, and may be activated by an activation mechanism (not shown) so that a user can sever a portion of fastening device 100. Cutting apparatus 622 may separate a segment or portion of fastening device 100 by breaking fastening device 100 at a segmentation point (as described above), or may be configured to cut through a section of fastening device 100 without a segmentation point.

### Example of Operation

In operation, and in connection with the embodiment(s) shown in FIGS. 1-5, a segment of coils 140 may first be inserted into the tube 172 of the delivery mechanism 170 so as to load the tube 172. Depending on the desired length of the fastening device 140, more than one segment of coils 140 may need to be used. Segmentation points 155 may be used to couple adjacent segments of coils 140. Alternatively, a relatively long piece of stock body portion 110 may be broken at a segmentation point and loaded into tube 172. Once the tube 172 is loaded, the tube 172 may be positioned adjacent a site of delivery.

A sufficient rotational force may then be applied to advance coils 140 though the grooves of the tube 172. As the rotational force continues to be applied, the coils 140 thereafter, may advance along track 180, and bias to an actuated position 150 where the coils 140 are spaced apart. Thereafter, the distal end 130 of the coils 140 may penetrate through layers of tissue to be fastened. The coils 140 may continue to be directed across the layers of tissue so that the body continues to advance into or across the tissue in a direction substantially transverse to the surface of the tissue, and so that the tissue situated between the coils is positioned substantially transverse to the body portion 110. The process may be repeated until the fastening device 100 has reached a desired depth. Once in place, the coils 140 may be allowed to revert to their resting position 160 so as to compress and pinch tissue between the coils 140. In some instances, as the coils compress, they may pinch multiple layers of tissue together, or may hold an object, such as a mesh, against the tissue. Following insertion of the fastening device 100, slight angulation of the distal end 174 of the tube 172 may disrupt or break the segmentation point 155 that hold adjacent segments of coils 140 to remove excess length from the just-placed fastening device, and to allow subsequent delivery and placement of a subsequent fastening device 100 from tube 172. Additionally/alternatively, segmentation point 155 may be broken by advancing a surgical instrument to the site and manually breaking segmentation point 155.

In another embodiment, in accordance with FIGS. 6a-c, a fastening device 100 may be loaded into driver 602 so that device 100 is accommodated in pathway 616 of helical member 606 to allow fastening device 100 to be held in an actuated state. Thereafter, helical member 606 may be driven into the tissue to be fastened by a rotational force, allowing the fastening device 100 to penetrate into the tissue along with the helical member 606. Fastening device 100 may then be held in place, while the helical member 606 is rotated in an opposite direction and withdrawn from the tissue. Upon exiting from the helical member 606, the fastening device 100 is allowed to revert to a relaxed state to pinch the tissue between adjacent coils for fastening the layers of tissue. A cutting apparatus may then cut the fastening device 100, so that the portion of the fastening device 100 that has been delivered into the tissue can remain in the tissue while the driver mechanism is removed.

While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains, and as fall within the scope of the appended claims.

What is claimed is:

1. A fastening device comprising:
a body portion designed to bias in the presence of an applied force between an actuated state for delivery into tissue to be fastened, and a quiescent state for fastening the tissue;
a penetration portion at a distal end of the body portion to permit initial penetration of the body portion into the tissue at a site of fastening; and
a segmentation point on the body to permit removal of excess body portion from the site of fastening.

2. A fastening device as set forth in claim 1, wherein the body is defined by a plurality of coils.

3. A fastening device as set forth in claim 2, wherein the plurality of coils can spread from one another to permit delivery of the body into the tissue.

4. A fastening device as set forth in claim 2, wherein the plurality of coils can compress toward one another to pinch the tissue between adjacent coils.

5. A fastening device as set forth in claim 1, wherein the body portion is constructed from a material that is biocompatible, bioresorbable, biodegradable, or a combination thereof.

6. A fastening device as set forth in claim 5, wherein the body portion is constructed from metal, metal alloy, polymer, molded plastic, metal-polymer blend, or a combination thereof.

7. A fastening device as set forth in claim 1, wherein the segmentation point includes a coupling mechanism to hold a plurality of body portions in axial alignment to extend the body portion lengthwise.

8. A fastening device as set forth in claim 1, wherein the segmentation point can be severed in order to separate the body portion into a plurality of segments.

9. A fastening device as set forth in claim 1, wherein the segmentation point can be situated at predetermined locations along the body portion to allow removal of segments of various sizes from the body portion

10. A fastening device comprising:
a body portion designed to bias, in the presence of an applied force, between an actuated state for delivery into tissue, and a quiescent state for fastening the tissue;
a penetration portion at the distal end of the body portion to permit initial penetration of the body portion into the tissue at a site of fastening; and
a plurality of coils defining the body portion and arranged so that tissue can be compressed between adjacent coils in a manner transverse to the body portion.

11. A fastening device as set forth in claim 10, wherein the plurality of coils can spread from one another to permit delivery of the body into tissue.

12. A fastening device as set forth in claim 10, wherein the plurality of coils can compress toward one another to pinch the tissue between adjacent coils.

13. A fastening device as set forth in claim 10, wherein the body portion is constructed from a material that is biocompatible, bioresorbable, biodegradable, or a combination thereof.

14. A fastening device as set forth in claim 13, wherein the body portion is constructed from metal, metal alloy, polymer, molded plastic, metal-polymer blend, or a combination thereof.

15. A fastening device as set forth in claim 10, further comprising at least one segmentation point situated along the body, to permit removal of excess body portion from the site of fastening.

16. A system for fastening tissue comprising:
a fastening device designed to bias between an actuated state for delivering into tissue and a quiescent state for fastening the tissue;
a pathway along which the fastening device can be accommodated for subsequent delivery into the tissue; and
an activation mechanism along the pathway to bias the fastening device into an activated state for delivering into the tissue.

17. A system as set forth in claim 16, wherein the pathway is a passage within a rigid helical member, the rigid helical member designed to rotate into tissue in the presence of a rotational force

18. A system as set forth in claim 17, wherein the activation mechanism is a pitch of the helical member configured to place the fastening device in an activated state when the fastening device is accommodated within the passage.

19. A system as set forth in claim 16 wherein the pathway is a tube for directing the fastening device to a site of fastening.

20. A system as set forth in claim 19 wherein the activation mechanism is a track situated within a distal end of the tube, the track having a pitch designed to place the fastening device in the activated state in the presence of a rotational force.

21. A method of fastening comprising:
biasing a fastening device into an activated state;
directing the device, in the activated state, into tissue to be fastened; and
allowing the fastening device to revert to a quiescent state to secure the tissue to be fastened between portions of the fastening device.

22. A method as set forth in claim 21, wherein the step of biasing includes advancing the device along a pathway that places the fastening device in the activated state.

23. A method as set forth in claim 22, wherein the step of directing includes applying a rotational force to the fastening device.

24. A method as set forth in claim 21, wherein the step of biasing includes accommodating the fastening device within a pathway in a rigid helical member having a pitch configured to place the fastening device in the activated state.

25. A method as set forth in claim 24, wherein the step of directing include applying a rotational force upon the rigid helical member so that the rigid helical member, and the fastening device therewith, are advanced into the tissue in a rotational manner.

26. A method as set forth in claim 24, wherein the step of allowing includes locking the fastening device in place while the helical member is counter-rotated, so that the fastening device remains within the tissue while the helical member is removed.

27. A method as set forth in claim 21, wherein the step of allowing includes pinching multiple layers of tissue together between the portions of the fastening device.

28. A method as set forth in claim 21, wherein the step of allowing includes pinching at least one layer of tissue and a foreign object together between the portions of the fastening device.

29. A method as set forth in claim 21, further comprising separating a segment of the fastening device that has been delivered into the tissue from a body of the fastening device, thus allowing the segment to remain in the tissue as the remainder of the body is withdrawn.
